(19) United States
(12) Patent Application Publication
(10) Pub. No.: US 2017/0184616 A1
(43) Pub. Date: Jun. 29, 2017

(54) SEPARATING AND QUANTIFYING THIAMINE PYROPHOSPHATE AND PYRIDOXAL 5-PHOSPHATE IN HUMAN WHOLE BLOOD

(71) Applicant: Micromass UK Limited, Wilmslow (GB)

(72) Inventors: Robert M. Wardle, Manchester (GB); Lisa Jane Calton, Stockport (GB)

(21) Appl. No.: 15/381,638

(22) Filed: Dec. 16, 2016

Related U.S. Application Data
(60) Provisional application No. 62/268,190, filed on Dec. 16, 2015.

(51) Int. Cl. G01N 33/82; B01D 15/32; C12Q 1/02

(52) U.S. Cl. CPC G01N 33/82; C12Q 1/02; B01D 15/325; G01N 2560/00

(57) ABSTRACT

The present disclosure provides robust, high-throughput, and clinically applicable methods for simultaneously separating and quantifying the biologically active forms of Vitamin B1 (TPP) and Vitamin B6 (PLP) from human whole blood.

### REFERENCE TO RELATED APPLICATIONS

This application claims priority to U.S. Provisional Application No. 62/268,190, filed Dec. 16, 2015, the entire contents of which are incorporated herein by reference.

### TECHNICAL FIELD

The present disclosure generally relates to methods for separating and quantifying Thiamine Pyrophosphate (TPP, Vitamin B1) and Pyridoxal 5-Phosphate (PLP, Vitamin B6) from human whole blood.

### BACKGROUND

Vitamin B1 (Thiamine) is a water soluble coenzyme that has a role in nervous function, the metabolism of carbohydrates and fatty acids, and is vital for normal growth and development. Thiamine is metabolized by Thiamine Pyrophosphokinase+ATP into its physiologically active form, Thiamine Pyrophosphate (TPP). Vitamin B6 is a group of three vitamins; Pyridoxal, Pyridoxine and Pyridoxamine. The biologically active form of Vitamin B6 is Pyridoxal 5-Phosphate (PLP), a coenzyme that is involved in approximately one hundred enzymatic reactions, including the synthesis and breakdown of amino acids, phospholipids and glycogen.

The analysis of both PLP and TPP is important to diagnose potential nutritional deficiency which often occurs in elderly people due to malnutrition, in severe alcoholism, and in gastrointestinal compromise due to surgery or disease. This can lead to conditions such as e.g., Beriberi, Wernicke-Korsakoff syndrome, alterations of the skin, neurological disorders, anemias, and increases in homocysteine (hyperhomocysteinemia). PLP and TPP have traditionally been analyzed independently using high performance liquid chromatography (HPLC) consisting of separate HPLC methods where total run times for each method can be up to 20 min long. In addition, pre-column derivatization of the PLP and TPP, such as with fluorescence-based detection, is also usually required. These methods are not realistic in a high-throughput clinical setting because they are laborious, costly, and typically require the use of toxic reagents such as potassium ferricyanide.

### SUMMARY

In general, the present disclosure relates to robust, high-throughput, and clinically applicable methods for simultaneously separating and quantifying the biologically active forms of Vitamin B1 (TPP) and Vitamin B6 (PLP) from human whole blood.

In one aspect, the disclosed methods comprise a single HPLC run from a sample of human whole blood without the need for pre-column derivatization. See e.g., FIG. 1. Thus, in one aspect, the methods disclosed herein eliminate the need for multiple HPLC runs and the use of toxic derivatization reagents.

In another aspect, the methods described herein minimize the elution time between the TPP and PLP on the chromatography column (e.g., less than 20 seconds apart from one another). See e.g., FIG. 1. Thus, in one aspect, this allows most of the column waste to be diverted from the mass spectrometer (e.g., in the case of quantification), thereby minimizing effects from early eluting interferences, which, as a result, improves detection sensitivity and accuracy as well as method robustness.

In yet another aspect, the methods described herein comprise short run times (e.g., total run times of 3.5 minutes or less, such as 3.2 minutes or less) and optimized retention times for TPP and PLP (e.g., tR of 45 seconds or greater. See e.g., FIG. 1. Thus, in one aspect, this significantly reduces the amount of solvents used, lowers cost(s), and allows for a high throughput of samples to be analyzed.

In yet another aspect, the methods described herein implement in certain aspects a mass spectrometer analysis of TPP and PLP where there is a single sample including a first known quantity of TPP and a first known quantity of PLP, and where the first known quantity of TPP, the known quantity of PLP, and the PLP and TPP from simultaneous separation from whole blood are each distinguishable within the single sample by mass spectrometry. Thus, in one aspect, this greatly reduces analysis times and eliminates the inefficiency of conventional calibration that uses multiple calibrators independently. See e.g., WO 2012/170549.

### BRIEF DESCRIPTION OF THE FIGURES

FIG. 1 depicts HPLC chromatograms of the simultaneous separation of PLP and TPP using the methods described herein.

FIG. 2 illustrates the signal to noise ratio of TPP and PLP from a low patient sample using the methods described herein.

FIG. 3 illustrates the calibration lines obtained from whole blood calibrators using the methods described herein.

### DETAILED DESCRIPTION OF CERTAIN EMBODIMENTS

In a first exemplary embodiment, provided is a method of quantifying both native Thiamine Pyrophosphate (TPP) and native Pyridoxal 5-Phosphate (PLP) from a single sample of human whole blood using mass spectrometry, comprising eluting TPP and PLP at time of less than 20 seconds apart from one another from a liquid chromatography column; generating a mass spectrometry signal during elution of the TPP and PLP; and quantifying the TPP and PLP using one or more calibration standards.

As used herein, the terms “native Thiamine Pyrophosphate (TPP)” and “native Pyridoxal 5-Phosphate (PLP)” mean TPP and PLP which have not been subjected to or undergone pre-column derivatization. It is to be understood that pre-column derivatization refers to any methods that are employed prior to analysis which chemically modify TPP, PLP, or both. Thus, sample preparation methods which does not involve chemical modification of TPP and PLP (e.g., precipitation, dilution, extraction, filtering, centrifugation, drying, and the like) do not constitute pre-column derivatization methods.

As used herein, calibration standards, also referred to as calibrators, mean a standard or reference material that contains a known amount of TPP or PLP, or both. These known amounts can then be used to generate a calibration line to which the amount of TPP and/or PLP can be determined from a sample containing an unknown amount. The use of calibration standards to determine unknown concentrations is known in the art. For example, for each calibrator standard the ratio of the peak area of TPP to the peak area of its corresponding internal standard is calculated. The determined response ratio (y) is plotted against the calibrator standard concentration (x) for TPP. A line of best fit (this can be linear, or quadratic) is drawn through all the calibrators and the equation of the line is determined, for example a linear line of best fit equation: y=0.00619507*x+0.0442534. Using the determined equation of the calibration line, the TPP concentration in an unknown sample (x) can be calculated by rearranging the equation: x=(y−0.0442534)÷0.00619507 and determining the response ratio in the unknown sample (for example y=0.6). Therefore, x=(0.6−0.0442534)÷0.00619507; x=89.7. Thus, the concentration of the unknown sample is 89.7 nmol/L. The concentration of PLP is determined e.g., in the same manner.

It is to be understood that while not mentioned in detail, other methods for determining the amount of TPP and/or PLP from a sample containing an unknown amount using calibration standards are encompassed. Thus, such determinations are not limited to those that require the use of mathematical equations. For example, unknown amounts can be determined by simply reading the response ratios alone, by reading the graph or calibrations lines, or the like.

Calibrators used by the methods described herein can be prepared using a number of matrices, e.g., by i) human whole blood augmented with TPP and PLP and/or diluted with 0.01M phosphate buffered saline solution (pH 7.4), which may contain 1% human serum albumin or 1% bovine serum albumin to generate the calibrators over a desired measuring range; by ii) TPP and PLP depleted whole blood augmented with TPP and PLP to generate the calibrators over a desired measuring range; by iii) surrogate matrix calibrators prepared from 0.01M phosphate buffered saline solution (pH 7.4) which may contain 1% human serum albumin or 1% bovine serum albumin and augmented with TPP and PLP to generate the calibrators over a desired measuring range; by iv) surrogate analyte calibrators prepared in human whole blood by the addition of a different stable labeled isotope to that of the internal standard for TPP and PLP to generate a surrogate analyte calibration curve for TPP and PLP over the desired measuring range; or by v) a single sample approach where one or more calibration standards (each of which having a different mass and/or fragmentation pattern) are contained in the same sample as the TPP and PLP and are eluted from the liquid chromatography column See e.g., WO 2012/170549.

Thus, in a second exemplary embodiment, quantifying the TPP and PLP using one or more calibration standards comprises augmenting human whole blood with TPP and/or PLP, and optionally diluting the human whole blood with phosphate buffered saline to generate the one or more calibration standards over a measuring range to which the amount of the TPP and PLP eluted from the column can be determined by mathematical expression, wherein the remaining features are as described in the first exemplary embodiment. Alternatively, quantifying the TPP and PLP using one or more calibration standards comprises augmenting TPP and PLP depleted whole blood with TPP and PLP to generate the one or more calibration standards over a measuring range to which the amount of the TPP and PLP eluted from the column can be determined by mathematical expression, wherein the remaining features are as described in the first exemplary embodiment. In another alternative, quantifying the TPP and PLP using one or more calibration standards comprises the use of surrogate matrix calibrators prepared from phosphate buffered saline solution optionally containing human serum albumin or bovine serum albumin and optionally augmented with TPP and PLP to generate the one or more calibration standards over a measuring range to which the amount of the TPP and PLP eluted from the column can be determined by mathematical expression, wherein the remaining features are as described in the first exemplary embodiment. In another alternative, quantifying the TPP and PLP using one or more calibration standards comprises the use of surrogate analyte calibrators prepared in human whole blood by the addition of a different stable labeled isotope to that of the internal standard for TPP and PLP to generate the one or more calibration standards over a measuring range to which the amount of the TPP and PLP eluted from the column can be determined by mathematical expression, wherein the remaining features are as described in the first exemplary embodiment. In yet another alternative, quantifying the TPP and PLP using one or more calibration standards comprises the placing one or more calibration standards of different masses and/or fragmentation patters in the same sample following the methods described in WO 2012/170549 together with the TPP and PLP eluted from the column, to which the amount of the TPP and PLP eluted from the column can be determined by e.g., mathematical expression, wherein the remaining features are as described in the first exemplary embodiment. Each of the calibrators described above may be provided in the form of a kit together with instructions to use said kit. Also, other non-mathematical based determinations for each of the above alternatives are contemplated and include e.g., reading the response ratios alone, reading the graph or calibrations lines, or the like.

Mathematical determinations of the amount of TPP and PLP eluted from the column when compared with the one or more calibrators described herein (such as the in the first and second exemplary embodiment) can be made following the methods described in e.g., WO 2012/170549. Again, other non-mathematical based determinations are contemplated and include e.g., reading the response ratios alone, reading the graph or calibrations lines, or the like.

In an third exemplary embodiment, also provided is a method of separating both native Thiamine Pyrophosphate (TPP) and native Pyridoxal 5-Phosphate (PLP) from whole blood comprising eluting TPP and PLP at time of less than 20 seconds apart from one another from a liquid chromatography column.

In a fourth exemplary embodiment, the methods described in the first, second, or third exemplary embodiment comprises eluting TPP and PLP at time of less than 17 seconds apart from one another from the liquid chromatography column Alternatively, the methods described in the first, second, or third exemplary embodiment comprises eluting TPP and PLP at time of less than 15 seconds apart from one another from the liquid chromatography column. In another alternative, the methods described in the first, second, or third exemplary embodiment comprises eluting TPP and PLP at time of less than 13 seconds apart from one another from the liquid chromatography column.

In a fifth exemplary embodiment, both the TPP and PLP have a retention time on the liquid chromatography column of 35 seconds or greater, wherein the remaining features are as described in the first, second, third, or fourth exemplary embodiment. Alternatively, both the TPP and PLP have a retention time on the liquid chromatography column of 40 seconds or greater, wherein the remaining features are as described in the first, second, third, or fourth exemplary embodiment. In another alternative, both the TPP and PLP have a retention time on the liquid chromatography column of 45 seconds or greater, wherein the remaining features are as described in the first, second, third, or fourth exemplary embodiment.

In a sixth exemplary embodiment, the total elution time of TPP and PLP does not exceed 3.5 minutes, wherein the remaining features are as described in the first, second, third, fourth, or fifth exemplary embodiment. Alternatively, the total elution time of TPP and PLP does not exceed 3.2 minutes, wherein the remaining features are as described in the first, second, third, fourth, or fifth exemplary embodiment. In another alternative, the total elution time of TPP and PLP does not exceed 3.0 minutes, wherein the remaining features are as described in the first, second, third, fourth, or fifth exemplary embodiment. In another alternative, the total elution time of TPP and PLP does not exceed 2.5 minutes, wherein the remaining features are as described in the first, second, third, fourth, or fifth exemplary embodiment. In yet another alternative, the total elution time of TPP and PLP does not exceed 2.0 minutes, wherein the remaining features are as described in the first, second, third, fourth, or fifth exemplary embodiment.

In a seventh exemplary embodiment, the liquid chromatography column is a reverse phase C18 HPLC column, wherein the remaining features are as described in the first, second, third, fourth, fifth, or sixth embodiment.

In an eighth exemplary embodiment, the liquid chromatography column is a reverse phase C18 HPLC column having a 3.5 μm particle size, wherein the remaining features are as described in the first, second, third, fourth, fifth, sixth, or seventh exemplary embodiment.

In a ninth exemplary embodiment, the liquid chromatography column is a reverse phase C18 HPLC column having a 3.5 μm particle size, a greater than 15% carbon load, and a pore size of greater than or equal to 100 Å, wherein the remaining features are as described in the first, second, third, fourth, fifth, sixth, seventh, eighth, or ninth exemplary embodiment.

In a tenth exemplary embodiment, the liquid chromatography column is a reverse phase C18 HPLC column having a 3.5 μm particle size, a 15% to 20% carbon load, and a pore size of 100 Å, wherein the remaining features are as described in the first, second, third, fourth, fifth, sixth, seventh, eighth, or ninth exemplary embodiment.

In an eleventh exemplary embodiment, the TPP and PLP are eluted from the liquid chromatography column using a mobile phase comprising water (H₂O), methanol (MeOH) and formic acid, wherein the remaining features are as described in the first, second, third, fourth, fifth, sixth, seventh, eighth, ninth, or tenth embodiment.

In a twelfth exemplary embodiment, the TPP and PLP are eluted from the liquid chromatography column using a mobile phase comprising H₂O together with 0.1% formic acid (v/v) and MeOH together with 0.1% formic acid (v/v), wherein the remaining features are as described in the first, second, third, fourth, fifth, sixth, seventh, eighth, ninth, tenth, or eleventh exemplary embodiment.

In a thirteenth exemplary embodiment, the TPP and PLP are eluted from the liquid chromatography column using H₂O together with 0.1% formic acid (v/v) and MeOH together with 0.1% formic acid (v/v), subject to a gradient comprising the following conditions:

| | | % Total of H₂O | | |
|---|---|---|---|---|
| | | together with | % Total of MeOH | |
| Time | Flow Rate | 0.1% formic acid | together with 0.1% | |
| (mm) | (mL/min) | (v/v) | formic acid (v/v) | Curve |
| 0 | 0.60 | 97 | 3 | Initial |
| 0.60 | 0.60 | 70 | 30 | 6 |
| 1.20 | 0.80 | 3 | 97 | 11 |
| 1.70 | 0.80 | 97 | 3 | 11 |
| 1.90 | 0.60 | 97 | 3 | 11 |

wherein the remaining features are as described in the first, second, third, fourth, fifth, sixth, seventh, eighth, ninth, tenth, eleventh, or twelfth exemplary embodiment.

In a fourteenth exemplary embodiment, the method described herein such as those described in the first, second, third, fourth, fifth, sixth, seventh, eighth, ninth, tenth, eleventh, twelfth, or thirteenth exemplary embodiment, further comprises the step of protein precipitating the TPP and PLP from whole blood prior to elution on the liquid chromatography column.

### EXEMPLIFICATION

Separation and Quantification of TPP and PLP Simultaneously from a Sample of Human Whole Blood

Equipment/Materials

ACQUITY UPLC I-Class Binary Solvent Manager

ACQUITY UPLC I-Class Sample Manager with Flow Through Needle

ACQUITY UPLC I-Class Active Column Heater

Xevo TQD

Methods

The LC required approximately 100 injections of a prepared whole blood sample to condition the column. Peaks were not visible in the first 5-10 samples for TPP or PLP and then their areas increased over the course of the injections. This was accommodated for when a new column was placed onto the system.

Mobile Phase A: H₂O+0.1% Formic Acid

Mobile Phase B: MeOH+0.1% Formic Acid

Purge Solvent: H₂O+0.1% Formic Acid

Seal Wash: 80:20 H₂O:MeOH

Wash Solvent: 80:20 H₂O:MeOH

Column: Symmetry C18, 2.1×100 mm, 3.5 μm (P/N WAT058965)

### Gradient Table

| | Flow Rate | | | |
|---|---|---|---|---|
| Time (min) | (mL/min) | % A | % B | Curve |
| 0.00 | 0.60 | 97 | 3 | Initial |
| 0.60 | 0.60 | 70 | 30 | 6 |
| 1.20 | 0.80 | 3 | 97 | 11 |
| 1.70 | 0.80 | 97 | 3 | 11 |
| 1.90 | 0.60 | 97 | 3 | 11 |

Inlet runtime: 2.0 min
Seal wash: 1.5 min
Low Pressure Limit: 1000 psi
High Pressure Limit: 18000 psi

### Autosampler Conditions

Needle volume: 30 μL (Optional extension loop)

Injection volume: 20 μL (Or range of 5 to 50 μL is also acceptable dependent upon mass spectrometer used)

Sample syringe volume: 100 μL (Optional larger volume syringe)

Sample temperature: 10° C.

Runtime: 2.0 min

Column temperature: 30° C.±2° C. alarm

Load Ahead: Disabled

Needle placement: 2 mm (when transferred or 10 mm when injecting straight off pellet)

### Xevo TQD Parameters

Approximate (optimized upon tuning)

Capillary: 1.0 kV (optimized upon tuning)

Source temperature: 150° C. (A range of 120 to 170° C. is also acceptable)

Desolvation temperature: 450° C. (A range of 300 to 600° C. is also acceptable)

Desolvation gas flow: 1000 L/Hr

Extractor: 3V

RF lens: 2.5V

Cone gas flow: 10 L/Hr

Inter-Scan Delay

MS inter-scan delay: 0.02 sec

Polarity/mode switch inter-scan delay: 0.02 sec

Inter-channel delay: 0.01 sec

### MRM Transitions

Approximate (optimized upon tuning)

| | Precursor | Product | Cone | Collision |
|---|---|---|---|---|
| Compound | (m/z) | (m/z) | (V) | (kV) |
| PLP (Quan) | 248.0 | 150.0 | 20* | 14 |
| PLP (Qual) | 248.0 | 94.0 | 20* | 28 |
| [²H₃]-PLP | 251.0 | 153.0 | 20* | 14 |
| TPP (Quan) | 425.0 | 122.0 | 30 | 22 |
| TPP (Qual) | 425.0 | 304.0 | 30 | 16 |
| [²H₃]-TPP | 428.0 | 125.0 | 30 | 22 |

*Cone Voltage not optimized due to an interfering compound at higher cone voltages. Value set to 20 V

The quantifier ions typically give better peak response, however other qualifier ions may be selected if deemed more suitable.

Dwell time for all compounds: 0.025 sec (Auto Dwell not selected)

The solvent delay and divert settings allow for the first 0.75 min of the run and the column wash to be diverted to waste. Given the higher flow rate of 0.6 mL/min, this allows for a good portion of the early eluting interferences and those eluting in the column wash to not enter the MS system. After the column wash, the mobile phase flow is sent back into the MS system to wash any TCA that may still be present in the MS system.

| Time (min) | Event | Action |
|---|---|---|
| 0.00 | Flow State | Waste |
| 0.00 | Solvent Delay | Begin |
| 0.75 | Solvent Delay | End |
| 0.75 | Flow State | LC |
| 1.35 | Flow State | Waste |
| 1.35 | Solvent Delay | Begin |
| 1.75 | Flow State | LC |

Scan time: 2.0 min

Sample Preparation

50 uL of internal standard working solution (approx 200 ng/mL (470 nmol/L) of [²H₃]-Thiamine Pyrophosphate & 100 ng/mL (400 nmol/L) of [²H₃]-Pyridoxal 5-Phosphate) was aliquoted into a 2 mL Square 96-Well Plate.

While mixing the plate, 50 μL of whole blood sample was added and mixing was left for at least 30 min*. In one alternative, while mixing the plate, 50 μL of whole blood sample was added and mixing was left for at least 30 seconds. *The mixing times were not optimized after the sample was added to the internal standard and after the trichloroacetic acid was added for the current method. A time course experiment could be performed to possibly reduce the mixing times.

While continuing to mix the plate, 400 μL of 200 g/L (20% (w/v)) of trichloroacetic acid_{(aq)} was added and mixing was left for at least 1 hr*. Alternative amounts of trichloroacetic acid can be added during sample preparation and include e.g., at least 5%, at least 10%, at least 15%, at least 20%, at least 25% (w/v), or higher.

The use of trichloroacetic acid in the sample preparation was found to negate the need for ion pairing reagents to be used in the mobile phase of the LC system. This effectively removes the risk of contamination to the LC-MS/MS system when using such additives. In addition, when injecting a minimum of 10 μL of a final diluent concentration of ≧50 g/L (≧5% (w/v)) of trichloroacetic acid, equating to an on column concentration of ≧0.5 mg, the negatively charged portion of the acid may act by binding to the positively charged sites in the column and system flow path (Fe⁺ and silanol groups). As a result, there were fewer charged sites remaining in the analytical system flow path and therefore the negatively charged phosphate groups present on thiamine pyrophosphate and pyridoxal 5-phosphate do not interact, preventing any unwanted secondary interactions during chromatographic separation.

Once mixing was complete, the sample was centrifuged at 5,000 g for 5 min at 4° C.

The samples were transferred into a 1 mL square collection plate for injection on the UPLC/MS/MS (Needle placement 2 mm) or were injected directly off the pellet (Needle placement 10 mm). In one alternative, the supernatant from the mixed sample was transferred into a 1 mL round 96 well collection plate for injection on the UPLC/MS/MS.

Results

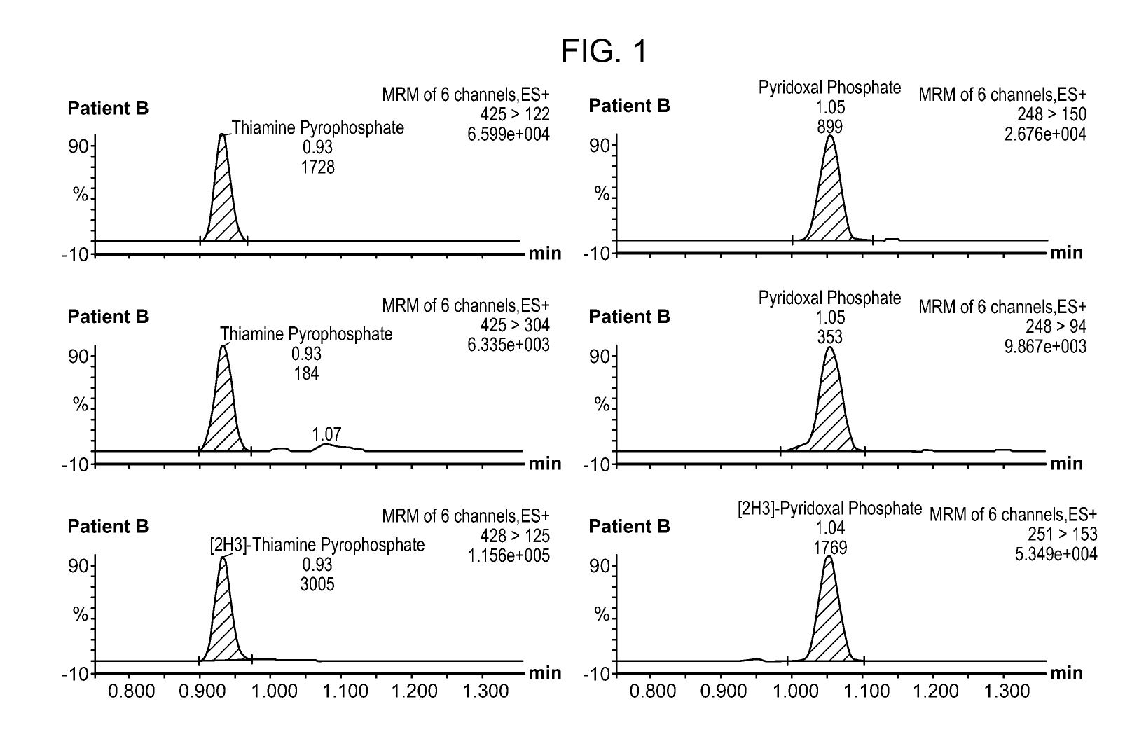

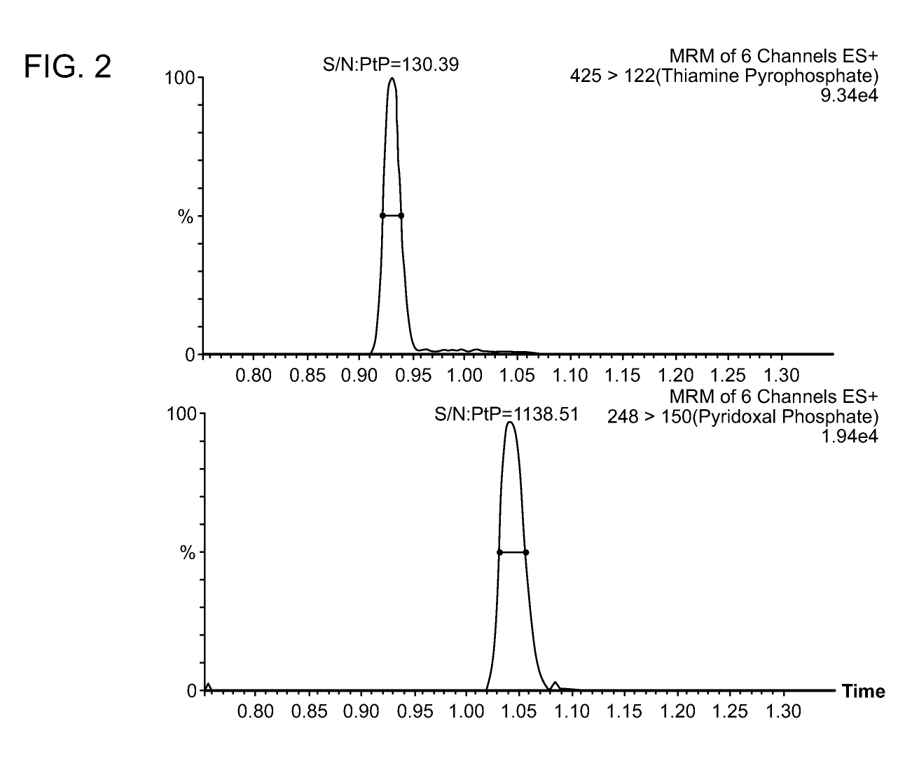

The chromatograms in FIG. 1 show the results of a low level sample: TPP approx 70 nmol/L, PLP approx 45 nmol/L. As shown by FIG. 2, a good signal to noise ratio was obtained from the low patient samples analyzed. This is due to the low amount of noise seen within all samples analyzed. TPP S:N ratio=125 (peak to peak) at 70 nmol/L PLP S:N ratio=1019 (peak to peak) at 45 nmol/L.

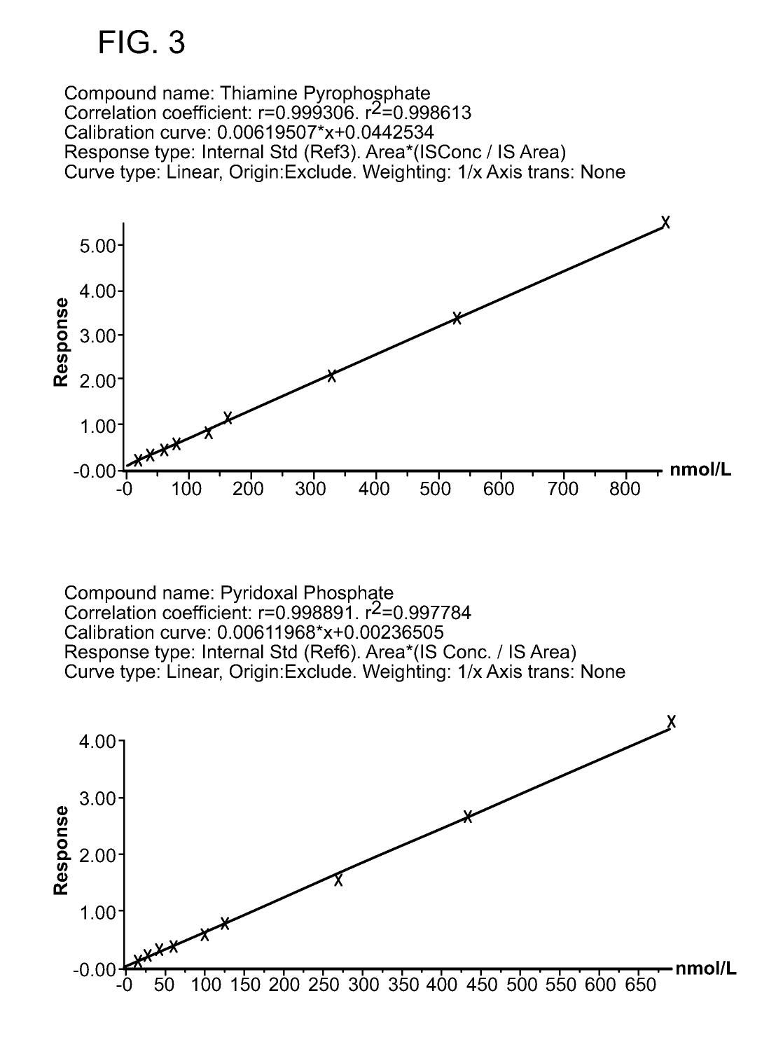

Calibration lines are shown in FIG. 3. Good r² values (linearity) were obtained from whole blood calibrators (also diluted in PBS/BSA to obtain concentrations lower than approx 100 nmol/L). The following calibration ranges were used in the 5 day imprecision run. TPP: 22.5-867.7 nmol/L. PLP: 15.1-691.3 nmol/L. In some instances, the gradient of the whole blood PLP calibration line reduced over the course of the 5 day imprecision testing. Without wishing to be bound by theory, it is thought that this could be due to a stability issue, either with freeze/thaw cycling or by storing the samples at ambient temperature while performing the sample preparation. PBS/BSA calibrators were also run alongside during testing which showed a more consistent gradient, therefore these were used during the processing of the imprecision data for PLP.

Imprecision

Samples from 4 different individuals to best span the concentration range were selected. 5 preparations of each individual were prepared and run on each of 5 days to assess imprecision. Good total run imprecision and repeatability were obtained for TPP and PLP. Imprecision data of ≦5.3% and 9.6% respectively, for all patient concentration levels, were observed.

TPP-Whole Blood Calibrators

| | Mean | Repeatability | Total |
|---|---|---|---|
| Sample | (nmol/L) | (%) | (%) |
| Patient A | 73.3 | 5.3 | 5.3 |
| Patient B | 85.3 | 4.1 | 4.9 |
| Patient C | 114.4 | 4.5 | 4.6 |
| Patient D | 206.1 | 4.0 | 5.2 |

PLP-PBS/BSA Calibrators

| | Mean | Repeatability | Total |
|---|---|---|---|
| Sample | (nmol/L) | (%) | (%) |
| Patient A | 44.5 | 9.3 | 9.6 |
| Patient B | 87.2 | 6.1 | 7.0 |
| Patient C | 140.5 | 4.1 | 4.1 |
| Patient D | 307.8 | 5.6 | 6.3 |

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A method of quantifying both native Thiamine Pyrophosphate (TPP) and native Pyridoxal 5-Phosphate (PLP) from a single sample of human whole blood using mass spectrometry, comprising:
eluting TPP and PLP at time of less than 20 seconds apart from one another from a liquid chromatography column;
generating a mass spectrometry signal during elution of the TPP and PLP; and
quantifying the TPP and PLP using one or more calibration standards.

2. The method of claim 1, wherein quantifying the TPP and PLP using one or more calibration standards comprises augmenting human whole blood with TPP and/or PLP, and optionally diluting the human whole blood with phosphate buffered saline to generate the one or more calibration standards over a measuring range to which the amount of the TPP and PLP eluted from the column can be determined by mathematical expression.

3. The method of claim 1, wherein quantifying the TPP and PLP using one or more calibration standards comprises augmenting TPP and PLP depleted whole blood with TPP and PLP to generate the one or more calibration standards over a measuring range to which the amount of the TPP and PLP eluted from the column can be determined by mathematical expression.

4. The method of claim 1, wherein quantifying the TPP and PLP using one or more calibration standards comprises the use of surrogate matrix calibrators prepared from phosphate buffered saline solution optionally containing human serum albumin or bovine serum albumin and optionally augmented with TPP and PLP to generate the one or more calibration standards over a measuring range to which the amount of the TPP and PLP eluted from the column can be determined by mathematical expression.

5. The method of claim 1, wherein quantifying the TPP and PLP using one or more calibration standards comprises the use of surrogate analyte calibrators prepared in human whole blood by the addition of a different stable labeled isotope to that of the internal standard for TPP and PLP to generate the one or more calibration standards over a measuring range to which the amount of the TPP and PLP eluted from the column can be determined by mathematical expression.

6. The method of claim 1, wherein quantifying the TPP and PLP using one or more calibration standards comprises the use placing one or more calibration standards in the same sample together with the TPP and PLP eluted from the column, to which the amount of the TPP and PLP eluted from the column can be determined by mathematical expression.

7. The method of claim 1, wherein the one or more calibrators are provided in the form of a kit.

8. A method of separating both native Thiamine Pyrophosphate (TPP) and native Pyridoxal 5-Phosphate (PLP) from whole blood comprising eluting TPP and PLP at time of less than 20 seconds apart from one another from a liquid chromatography column.

9. The method of claim 1, comprising eluting TPP and PLP at time of less than 17 seconds apart from one another from the liquid chromatography column

10. The method of claim 1, comprising eluting TPP and PLP at time of less than 15 seconds apart from one another from the liquid chromatography column.

11. The method of claim 1, comprising eluting TPP and PLP at time of less than 13 seconds apart from one another from the liquid chromatography column

12. The method of claim 1, wherein both the TPP and PLP have a retention time on the liquid chromatography column of 35 seconds or greater.

13. The method of claim 1, wherein both the TPP and PLP have a retention time on the liquid chromatography column of 40 seconds or greater.

14. The method of claim 1, wherein both the TPP and PLP have a retention time on the liquid chromatography column of 45 seconds or greater.

15. The method of claim 1, wherein the total elution time of TPP and PLP does not exceed 3.5 minutes.

16. The method of claim 1, wherein the total elution time of TPP and PLP does not exceed 3.0 minutes.

17. The method of claim 1, wherein the total elution time of TPP and PLP does not exceed 2.5 minutes.

18. The method of claim 1, wherein the total elution time of TPP and PLP does not exceed 2.0 minutes.

19. The method of claim 1, wherein the liquid chromatography column is a reverse phase C18 HPLC column.

20. The method of claim 1, wherein the liquid chromatography column is a reverse phase C18 HPLC column having a 3.5 μm particle size.

21. The method of claim 1, wherein the liquid chromatography column is a reverse phase C18 HPLC column having a 3.5 μm particle size, a greater than 15% carbon load, and a pore size of greater than or equal to 100 Å.

22. The method of claim 1, wherein the liquid chromatography column is a reverse phase C18 HPLC column having a 3.5 μm particle size, a 15% to 20% carbon load, and a pore size of 100 Å.

23. The method of claim 1, wherein the TPP and PLP are eluted from the liquid chromatography column using a mobile phase comprising water (H₂O), methanol (MeOH) and formic acid.

24. The method of claim 1, wherein the TPP and PLP are eluted from the liquid chromatography column using a mobile phase comprising H₂O together with 0.1% formic acid (v/v) and MeOH together with 0.1% formic acid (v/v).

25. The method of claim 1, wherein the TPP and PLP are eluted from the liquid chromatography column using H₂O together with 0.1% formic acid (v/v) and MeOH together with 0.1% formic acid (v/v), subject to a gradient comprising the following conditions:

| | | % Total of H₂O | % Total of MeOH | |
|---|---|---|---|---|
| Time | Flow Rate | together with 0.1% | together with 0.1% | |
| (mm) | (mL/min) | formic acid (v/v) | formic acid (v/v) | Curve |
| 0 | 0.60 | 97 | 3 | Initial |
| 0.60 | 0.60 | 70 | 30 | 6 |
| 1.20 | 0.80 | 3 | 97 | 11 |
| 1.70 | 0.80 | 97 | 3 | 11 |
| 1.90 | 0.60 | 97 | 3 | 11. |

26. The method of claim 1, further comprising the step of protein precipitating the TPP and PLP from whole blood prior to elution on the liquid chromatography column

27. The method of claim 1, wherein prior to eluting TPP and PLP, the TPP and PLP are mixed with trichloroacetic acid.
